(19) United States
(12) Patent Application Publication
(10) Pub. No.: US 2021/0108009 A1
(43) Pub. Date: Apr. 15, 2021

(54) METHOD FOR PRODUCING ALICYCLIC ACRYLATE DERIVATIVE

(71) Applicant: SONGWON INDUSTRIAL CO., LTD., Ulsan (KR)

(72) Inventors: Dong Kyung PARK, Busan (KR); Seok Jin HWANG, Ulsan (KR); Seung Jin CHEON, Ulsan (KR); Gyu Sung LEE, Busan (KR)

(73) Assignee: SONGWON INDUSTRIAL CO., LTD.

(21) Appl. No.: 17/069,937

(22) Filed: Oct. 14, 2020

(30) Foreign Application Priority Data
Oct. 14, 2019 (KR) 10-2019-0126904
Oct. 12, 2020 (KR) 10-2020-0130995

(51) Int. Cl. C08F 20/06; C08L 33/10

(52) U.S. Cl. CPC C08F 20/06; G03F 7/0397; C08L 33/10

(57) ABSTRACT

Provided is a composition including an alicyclic acrylate derivative. A composition including an alicyclic acrylate derivative according to one embodiment of the present invention includes an alicyclic acrylate compound but does not include an amine derivative by-product.

### CROSS-REFERENCE TO RELATED APPLICATION

This application claims priority to and the benefit of Korean Patent Application Nos. 10-2019-0126904 filed on Oct. 14, 2019, and 10-2020-0130995 filed on Oct. 12, 2020, the disclosure of which is incorporated herein by references in their entirety.

### FIELD

The present invention relates to an alicyclic acrylate derivative compound and a method of producing an alicyclic acrylate derivative, and more particularly, to a method of producing an alicyclic acrylate derivative, which is capable of suppressing the formation of an amine derivative by-product.

### BACKGROUND

A photoresist is a light-sensitive material used for transferring an image to a substrate. When a substrate on which a photoresist coating layer is formed is exposed to light, a relief which enables the selective treatment of the substrate may be formed.

There are various types of photoresist synthesized by various methods, and among the various methods, a method of synthesizing an alicyclic acrylate derivative by reacting an alcohol compound and methacryloyl chloride is widely used.

Although the above reaction is generally carried out using triethylamine (TEA) as a neutralizing agent, in this case, there is a problem that various types of amine derivatives may be produced as by-products, which may adversely affect the quality of a product.

Therefore, there is an emerging need to develop a new method of producing an alicyclic acrylate derivative capable of improving the quality of a product by suppressing the production of an amine derivative.

### SUMMARY

The present invention is directed to providing a method of producing an alicyclic acrylate derivative, which is capable of suppressing the production of an amine derivative by-product.

One aspect of the present invention provides a composition including an alicyclic acrylate derivative, which includes an alicyclic acrylate derivative represented by Formula (II) synthesized by reacting a compound having an alicyclic hydrocarbon group and a compound represented by Formula (I) but does not include an amine derivative represented by Formula (III) or Formula (IV), which is a by-product.

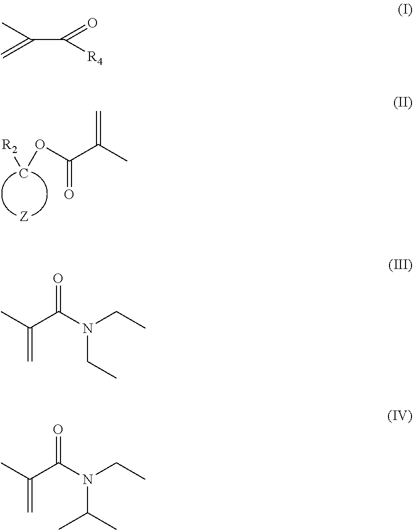

Here, R₂ is any one of a C1-C10 linear or branched alkyl group and an aromatic compound, R₄ is any one of a halogen element, alkoxy, and a (meth)acrylate, and Z is three to five carbon atoms forming an alicyclic hydrocarbon group.

According to one embodiment of the present invention, the compound having an alicyclic hydrocarbon group may be

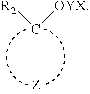

Here, X is a halogen element, and Y is a Group 1 or 2 metal element.

According to one embodiment of the present invention, the compound having an alicyclic hydrocarbon group may be synthesized by reacting a dianionic compound and a compound represented by the formula

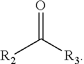

Here, R₂ is any one of a C1-C10 linear or branched alkyl group and an aromatic compound and R₃ is any one of a C1-C8 alkoxy, an anhydride, and a halogen element.

According to one embodiment of the present invention, the compound represented by the formula

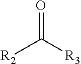

may be methyl isobutyrate.

According to one embodiment of the present invention, the compound having an alicyclic hydrocarbon group and the compound represented by Formula (I) may be reacted in the absence of TEA, which is a neutralizing agent, to synthesize the alicyclic acrylate compound represented by Formula (II).

Another aspect of the present invention provides a method of producing an alicyclic acrylate derivative, which includes: reacting a dianionic compound and a compound represented by Formula (V) and thus synthesizing a compound represented by Formula (VI)

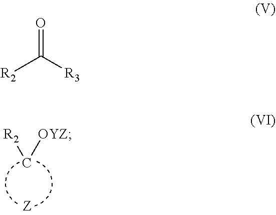

and

reacting the compound represented by Formula (VI) and a compound represented by Formula (VII) and thus synthesizing an alicyclic acrylate compound represented by Formula (VIII).

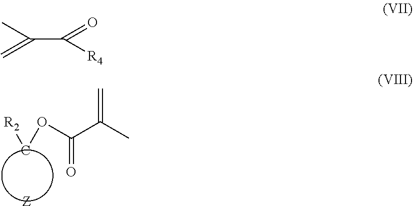

Here, R2 is any one of a C1-C10 linear or branched alkyl group and an aromatic compound, R3 is any one of a C1-C8 alkoxy, an anhydride, and a halogen element, R4 is any one of a halogen element, alkoxy, and a (meth)acrylate, X is a halogen group element, and Y is a Group 1 or 2 metal element.

According to one embodiment of the present invention, the compound represented by Formula (V) may be methyl isobutyrate.

According to one embodiment of the present invention, in the compound represented by Formula (VI), Z may be four carbon atoms forming an alicyclic hydrocarbon group, and R₂ may be isopropyl.

According to one embodiment of the present invention, a conversion rate to the compound represented by Formula (VI) may be 95% or more, and a conversion rate to the compound represented by Formula (VIII) may be 90% or more.

### DETAILED DESCRIPTION

Hereinafter, exemplary embodiments of the present invention will be described in detail with reference to the accompanying drawings. Advantages and features of the present invention and a method of achieving the advantages and features will become apparent with reference to the accompanying drawings and the exemplary embodiments described below in detail. However, the present invention is not limited to the exemplary embodiments to be described below but can be implemented in various different forms, and the exemplary embodiments are provided to make the disclosure of the present invention complete and fully inform the scope of the present invention to those of ordinary skill in the art to which the present invention pertains, and the present invention is only defined by the scope of the claims. Throughout the present specification, the same reference numerals refer to the same components.

Unless otherwise defined, all terms (including technical and scientific terms) used in the present specification may be used with meanings that can be commonly understood by those of ordinary skill in the art to which the present invention pertains. In addition, terms defined in commonly used dictionaries are not interpreted ideally or excessively unless explicitly defined specifically.

In addition, in the present specification, singular expressions include plural expressions unless the context clearly indicates otherwise. As used in the specification, “comprise,” “include,” “have,” “comprising,” “including,” and/or “having” do(es) not preclude the presence or addition of one or more other components, steps, operations, and/or elements besides the mentioned component, step, operation, and/or element.

A method of producing an alicyclic acrylate derivative according to one embodiment of the present invention includes synthesizing an alicyclic acrylate derivative from a compound having an alicyclic hydrocarbon group.

Hereinafter, a cyclization reaction in which a compound having an alicyclic hydrocarbon group is synthesized is referred to as a first reaction, and a reaction in which an alicyclic acrylate derivative is synthesized from the compound having an alicyclic hydrocarbon group is referred to as a second reaction.

The first reaction, which is a cyclization reaction, and the second reaction, which is a synthesis reaction of an alicyclic acrylate derivative, are sequentially performed. Specifically, the first reaction and the second reaction may be consecutively performed in one reaction vessel.

Hereinafter, the first reaction and the second reaction for producing an alicyclic acrylate derivative will be described in detail.

First Reaction: Cyclization Reaction

The cyclization reaction for producing an alicyclic acrylate derivative according to one embodiment of the present invention is carried out through a reaction between a dianionic compound and a metal cation.

Here, the dianionic compound may be represented by Chemical Formula 1 or Chemical Formula 2.

XY—R₁—YX [Chemical Formula 1]

Y—R₁—Y [Chemical Formula 2]

Here, X may be a halogen element, Y may be a Group 1 or 2 metal element, and R₁ may be any one of C3-C7 alkyl groups. Preferably, X is one of chlorine, bromine, and iodine, Y is one of lithium and magnesium, and R₁ is a C4 alkyl group.

When the dianionic compound is a compound represented by Chemical Formula 1, the dianionic compound may be a compound obtained by reacting any one of 1,4-dichlorobutane, 1,4-dibromobutane, and 1,4-diiodinebutane with a magnesium reagent.

The reaction may be represented by the following reaction scheme.

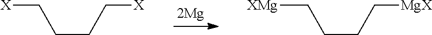

When the dianionic compound is a compound represented by Chemical Formula 2, the dianionic compound may be a compound obtained by reacting any one of 1,4-dichlorobutane, 1,4-dibromobutane, and 1,4-diiodinebutane with a lithium reagent.

The reaction may be represented by the following reaction scheme.

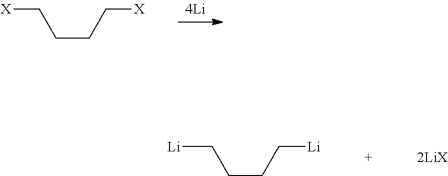

When the above dianionic compound reacts with a compound represented by Chemical Formula 3, the cyclization reaction occurs.

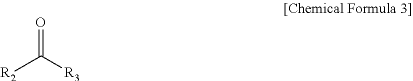

Here, R₂ may be any one of a C1-C10 linear or branched alkyl group and an aromatic compound, and R₃ may be any one of a C1-C8 alkoxy, an anhydride, and a halogen element.

Preferably, the compound represented by Chemical Formula 3 is methyl isobutyrate, which is represented by Chemical Formula 4.

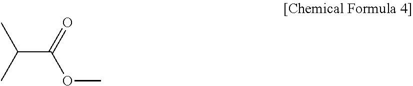

The compound obtained through the cyclization reaction may be represented by Chemical Formula 5.

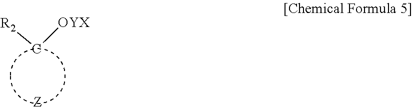

Here, Z represents a plurality of carbon atoms required to form a C3-05 alicyclic hydrocarbon group together with carbon atoms. Preferably, R2 is any one of isopropyl, methyl, ethyl, and t-butyl groups, Y is magnesium (Mg), Z is a C4 alicyclic hydrocarbon, and the cyclic compound represented by Chemical Formula 5 is 1-isopropyl-cyclopentanol (IPCPOH).

Meanwhile, according to one embodiment of the present invention, a conversion rate from the compound represented by Chemical Formula 1 or Chemical Formula 2, which is a starting material, to the cyclic compound represented by Chemical Formula 5, which is a product, may be 90% or more. Preferably, the conversion rate to the compound represented by Chemical Formula 5 is 95%.

The conversion rate refers to a mass fraction of the compound represented by Chemical Formula 5, as determined by treating the compound represented by Chemical Formula 5 with an acid and then subjecting the resultant to gas chromatography (GC) analysis.

Specifically, the conversion rate refers to a ratio obtained by dividing the mass of the compound represented by Chemical Formula 5 by the sum of the masses of methyl isobutyrate, the compound represented by Chemical Formula 5, and miscellaneous by-products.

When the cyclization reaction, which is the first reaction, is terminated, a synthesis reaction of an alicyclic acrylate derivative, which is the second reaction, is subsequently initiated in the same reaction vessel. The second reaction according to one embodiment of the present invention will be described below in detail.

Second Reaction: Synthesis Reaction of Alicyclic Acrylate Derivative

The compound represented by Chemical Formula 5, which is a product of the first reaction, that is, the cyclization reaction, is reacted with a compound represented by Chemical Formula 6.

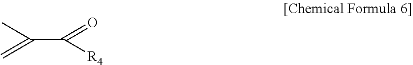

Here, R₄ may be one of a halogen element, alkoxy, and a methacrylate. Preferably, the compound represented by Chemical Formula 6 is one of an alkyl (meth)acrylate, di(meth)acrylic anhydride, and acryloyl chloride.

In the synthesis reaction of an alicyclic acrylate derivative, when the compound represented by Chemical Formula 5, hydrochloric acid, and the compound represented by Chemical Formula 6 are reacted, an esterification reaction occurs, and thereby an alicyclic acrylate derivative represented by Chemical Formula 7 is produced. In this case, a subsequent separation or fractional distillation process may be additionally performed to increase the purity of the compound represented by Chemical Formula 7.

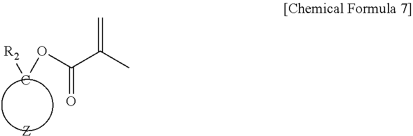

A conversion rate to the compound represented by Chemical Formula 7, which is a final product produced by the above-described method, may be 90% or more. Here, the conversion rate refers to a ratio obtained by dividing the mass of the compound represented by Chemical Formula 7, which is measured by the GC analysis method, by the sum of the masses of the compound represented by Chemical Formula 5, the compound represented by Chemical Formula 7, and miscellaneous by-products.

However, in the method of producing an alicyclic acrylate derivative according to the present invention, since an alcohol compound and methacryloyl chloride are not used as starting materials and TEA is not used as a neutralizing agent, the production of amine derivatives represented by Chemical Formula 8 and Chemical Formula 9 can be suppressed.

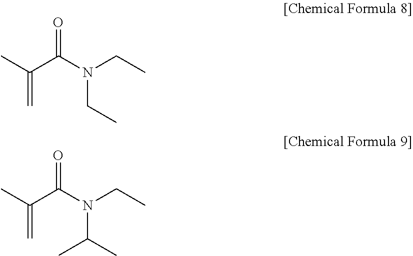

A composition including an alicyclic acrylate derivative produced according to one embodiment of the present invention may include the compound represented by Chemical Formula 8 or Chemical Formula 9 at 200 ppm or less. Preferably, the composition does not include the compound represented by Chemical Formula 8 or Chemical Formula 9.

Hereinafter, the present invention will be described in more detail through specific examples and comparative examples. However, compounds of the present invention are not limited to the following examples and comparative examples.

### Example 1: Method of Producing Alicyclic Acrylate Derivative of the Present Invention

After mixing Mg (0.50 mol, 12.2 g) and 120 g of anhydrous tetrahydrofuran (THF) thoroughly, 1,4-dichlorobutane (0.23 mol, 29.6 g) was added dropwise at 66° C. for two hours and then stirred at 66° C. for three hours to consume all of the 1,4-dichlorobutane. A Grignard reagent was prepared, and the temperature was lowered to 10° C. Methyl isobutyrate (0.17 mol, 17.0 g) was added dropwise for one hour and then stirred for one hour. After producing the compound represented by Chemical Formula 5 through the cyclization reaction, methacrylic anhydride (0.42 mol, 64.2 g) was added dropwise in-situ at a temperature of 20 to 25° C. for 0.5 hours and then reacted at 25° C. for two hours, and thereby 1-isopropylcyclopentyl methacrylate was obtained.

In this case, neither the compound represented by Chemical Formula 8 nor the compound represented by Chemical Formula 9 was detected as a by-product.

### Example 2: Method of Producing Alicyclic Acrylate Derivative of the Present Invention

After mixing Mg (0.50 mol, 12.2 g) and 240 g of anhydrous THF thoroughly, 1,4-dibromobutane (0.23 mol, 50.3 g) was added dropwise at a temperature of 45 to 55° C. for two hours and then stirred at a temperature of 45 to 55° C. for two hours to consume all of the 1,4-dibromobutane. A Grignard reagent was prepared, and the temperature was lowered to 0° C. Methyl isobutyrate (0.17 mol, 17.0 g) was added dropwise for one hour and then stirred for one hour. After producing the compound represented by Chemical Formula 5 through the cyclization reaction, methacrylic anhydride (0.42 mol, 64.2 g) was added dropwise in-situ at a temperature of 20 to 25° C. for 0.5 hours and then reacted at 25° C. for 10 hours, and thereby 1-isopropylcyclopentyl methacrylate was obtained.

In this case, neither the compound represented by Chemical Formula 8 nor the compound represented by Chemical Formula 9 was detected as a by-product.

### Comparative Example 1: Production Method Using Cyclic Ketone

An alicyclic acrylate derivative may also be produced through the following process:

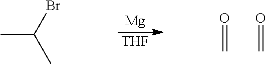

Specifically, after mixing Mg (0.25 mol, 6.1 g) and 180 g of anhydrous THF thoroughly, 2-bromopropane (0.25 mol, 30.7 g) was added dropwise at a temperature of 45 to 55° C. for one hour and then stirred at a temperature of 45 to 55° C. for two hours to consume all of the 2-bromopropane. A Grignard reagent was prepared, and the temperature was lowered to 0° C. A cyclic ketone (0.17 mol, 14.0 g) was added dropwise for 30 minutes and then stirred for 1 hour and 30 minutes. After producing the compound represented by Chemical Formula 5 (here, Y is Mg and X is bromine) through an addition reaction, methacrylic anhydride (0.21 mol, 32.9 g) was added dropwise in-situ at a temperature of 20 to 25° C. for 0.5 hours and then reacted at 25° C. for 10 hours, and thereby 1-isopropylcyclopentyl methacrylate was obtained.

Subsequently, the conversion rates and yields of the production method of the present invention and the method using a cyclic ketone were compared. The results are shown in Table 1.

TABLE 1

| | Conversion rate | |
|---|---|---|
| | (conversion %) to compound | |
| | represented by Chemical | |
| | Formula 5 | |
| | (In Chemical Formula 5, R₂ | |
| | is isopropyl, Z is C4 alicyclic | Rate of conversion to |
| | hydrocarbon, Y is Mg, and X | alicyclic acrylate derivative |
| | is chlorine or bromine) | of present invention |
| Example 1 | 95.0% | 92.1% |
| Example 2 | 96.8% | 90.1% |
| Comparative | 5.1% | 15.7% |
| Example 1 | | |

### Comparative Example 2: Production Method Using Alcohol Compound

An alicyclic acrylate derivative may also be produced through the following process:

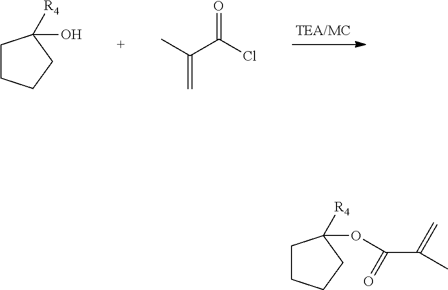

1-Isopropylcyclopentanol (1.36 mol, 175.0 g), methacryloyl chloride (2.05 mol, 214.0 g), and 1,050 g of methylene chloride were mixed thoroughly, and then the reactants were cooled. Subsequently, TEA (2.73 mol, 276.2 g) was added dropwise for 0.5 hours and then reacted overnight to consume all of the 1-isopropylcyclopentanol, and thereby 1-isopropylcyclopentyl methacrylate was obtained.

| | 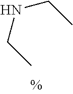 | 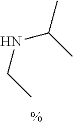 | IPCPMA Sample | 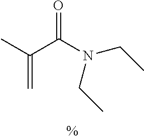 | 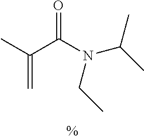 |
|---|---|---|---|---|---|
| TEA | 0.044 | 0.075 | Detected | 0.299 | 0.132 |
| | | | amount of | | |
| | | | amine derivative | | |

The amine derivatives, which are by-products, were detected, and amounts thereof were measured using a GC method under analysis conditions detailed below.

Analysis Conditions:

Capillary column: HP-5

Inlet: Initial temperature=150° C.; pressure=5.70 psi; split ratio=50:1; split flow=50.6 mL/min

Oven temperature: 80 to 290° C.

Detector: Flame ionization detector (FID) set at 310° C.

The above-described method of producing a compound for a photoresist according to one embodiment of the present invention has a high raw material conversion rate and a high yield, and is a simple and economical process due to not including a process of separating an alcohol.

Those of ordinary skill in the technical field related to the present invention will understand that the present invention can be implemented in a modified form without departing from the essential characteristics of the disclosure. Therefore, the disclosed methods should be considered from an explanatory point of view rather than a limiting point of view. The scope of the present invention is shown in the claims rather than the above description, and all differences within the scope of equivalents of the claims should be construed as being included in the present invention.

What is claimed is:

1. A composition comprising a compound of Formula (II), wherein the compound of Formula (II) is synthesized by reacting a compound having an alicyclic hydrocarbon group and a compound represented by Formula (I), and wherein the composition does not include an amine derivative represented by Formula (III) or Formula (IV), 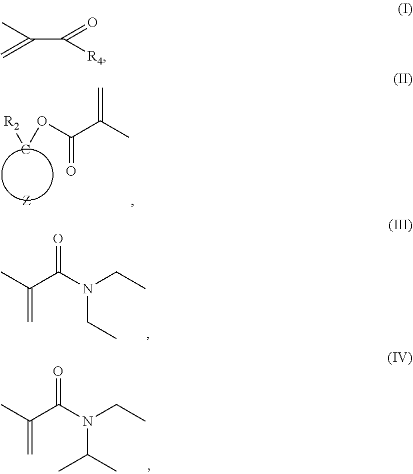
wherein R₂ is any one of a C1-C10 linear or branched alkyl group and an aromatic compound, R₄ is any one of a halogen element, alkoxy, and a (meth)acrylate, and Z is three to five carbon atoms forming an alicyclic hydrocarbon group.

2. The composition of claim 1, wherein the compound having an alicyclic hydrocarbon group is 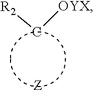
wherein X is a halogen element, and Y is a Group 1 or 2 metal element.

3. The composition of claim 2, wherein the compound having an alicyclic hydrocarbon group includes an alicyclic acrylate derivative synthesized by reacting a dianionic compound and a compound represented by a formula 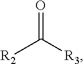
wherein R₂ is any one of a C1-C10 linear or branched alkyl group and an aromatic compound, and R₃ is any one of a C1-C8 alkoxy, an anhydride, and a halogen element.

4. The composition of claim 3, wherein the compound represented by the formula 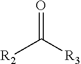
is methyl isobutyrate.

5. The composition of claim 1, wherein the compound having an alicyclic hydrocarbon group and the compound represented by Formula (I) are reacted in an absence of triethylamine, which is a neutralizing agent, and thus the alicyclic acrylate compound represented by Formula (II) is synthesized.

6. A method of producing an alicyclic acrylate derivative, comprising:
reacting a dianionic compound and a compound represented by the following Formula (V) and thus synthesizing a compound represented by the following Formula (VI) 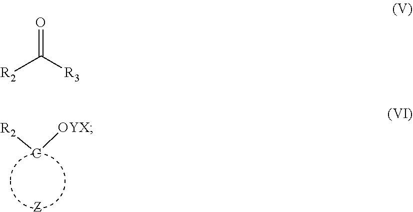
and
reacting the compound represented by Formula (VI) and a compound represented by Formula (VII) and thus synthesizing an alicyclic acrylate compound represented by Formula (VIII) 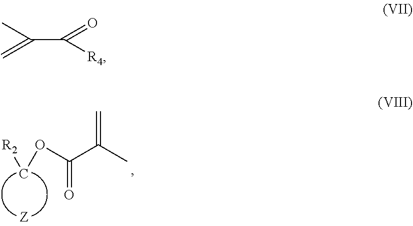
wherein R₂ is any one of a C1-C10 linear or branched alkyl group and an aromatic compound, R₃ is any one of a C1-C8 alkoxy, an anhydride, and a halogen element, R₄ is any one of a halogen element, alkoxy, and a (meth)acrylate, X is a halogen group element, and Y is a Group 1 or 2 metal element.

7. A method of protonating a compound represented by Formula (VI) with a hydrogen atom and thus synthesizing an alicyclic alcohol (IX),

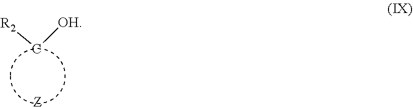

8. The method of claim 6, wherein the compound represented by Formula (V) is methyl isobutyrate.

9. The method of claim 6, wherein in the compound represented by Formula (VI), Z is four carbon atoms forming an alicyclic hydrocarbon group, and R₂ is isopropyl.

10. The method of claim 6, wherein a conversion ratio to the compound represented by Formula (VI) is 95% or more, and a conversion ratio to the compound represented by Formula (VIII) is 90% or more.
